(19) United States
(12) Patent Application Publication
(10) Pub. No.: US 2025/0011679 A1
(43) Pub. Date: Jan. 9, 2025

(54) PROCESS FOR REUSING LYSO-GUM, USED IN THE PRETREATMENT OF DEGUMMED PLANT OILS FOR SUBSEQUENT ENZYMATIC TREATMENT AND BIODIESEL TRANSESTERIFICATION

(71) Applicants: Jean Paulo de OLIVEIRA, Ijuí (BR); Luciano de MORAES, Ijuí (BR)

(72) Inventors: Jean Paulo de OLIVEIRA, Ijuí (BR); Luciano de MORAES, Ijuí (BR)

(21) Appl. No.: 18/711,595

(22) PCT Filed: Dec. 16, 2021
(86) PCT No.: PCT/BR2021/050555

§ 371 Date: May 19, 2024

(51) Int. Cl. C11B 3/00; C12N 9/20; C12P 7/62

(52) U.S. Cl. CPC C11B 3/003; C12N 9/20; C12P 7/62; C12Y 301/01032

(57) ABSTRACT

The process of reuse lysogoma, applied in the pre-treatment of already degummed oil for subsequent PLA treatment and transesterification of biodiesel object of the present invention comprises a pre-treatment step through the reuse of lysogoma originating from the PLA enzymatic treatment in the oil already degummed. The use of lysogoma containing active enzymes resulting from the enzymatic treatment (11) on the already degummed oil (40) aims to reduce the levels of phosphorus, calcium and magnesium. In short, the use of lysogoma (10) is presented as a pre-treatment as it aims to reduce the contents of phosphorus, calcium and magnesium present in the degummed oil. Furthermore, this new process impacts the reuse of a byproduct with no commercial value that helps to significantly reduce the contents of phosphorus, calcium and magnesium, which negatively interfere in the processes, especially in the PLA enzymatic treatment and transesterification steps for biodiesel production.

### FIELD OF THE INVENTION

The present invention describes a process for reusing lysogoma [sludge], which consists of a residue from the enzymatic treatment of PLA (Phospholipase A). The residue resulting from the treatment process of already degummed oil, industrially known as lysogoma, is applied in a pre-treatment of already degummed oils for subsequent treatment and transesterification of biodiesel. In short, the use of lysogoma presents as a pre-treatment, as it aims to reduce the contents of phosphorus, calcium and magnesium present in the degummed oil through the action of active enzymes present in the lysogoma and through intermolecular interactions.

This new process results in the reuse of a by-product with no commercial value that helps to significantly reduce the contents of phosphorus, calcium and magnesium. High contents of these chemical elements negatively interfere in the modification processes of vegetable oils, especially in the treatment and transesterification steps for biodiesel production.

### BACKGROUND OF THE INVENTION

Currently, industries treat already degummed oil through the PLA enzymatic method with phospholipase activity. Phospholipases (A) are enzymes that cleave the fatty acid in position two of phospholipids, hydrolyzing the bond between the fatty acid and the glycerol molecule. This particular phospholipase specifically recognizes the bond to be cleaved.

The enzyme PLA (Phospholipase A) selectively hydrolyzes the fatty acid opposite the phosphate functional group in the glycerol structure, producing a lyso-phospholipid and a fatty acid. The lyso-phospholipid molecule that has lost a hydrophilic functional group and the remaining alcohol group at the reaction site is hydrophilic. From the two hydrophilic sites, the lyso-phospholipid molecule becomes soluble in water and loses its emulsification properties. Therefore, the PLA (Phospholipase A) treatment process for already degummed oil reduces refining losses.

The reaction of the phospholipase enzyme with phospholipids can take many hours depending on reaction variables such as pH, temperature, relative concentrations and mixing conditions. PLA enzymatic treatment is typically carried out on vegetable oils and compositions that have first been subjected to degumming to obtain a product that has a low enough phosphorus level for the intended purposes.

The PLA enzymatic treatment in the already degummed oil is carried out aiming reducing the remaining contents of phosphorus, calcium and magnesium.

In conventional PLA enzymatic treatment, the already degummed oil is heated by water vapor from 55 to 70° C. and then 3-3.5% of water and 60-80 ppm of enzyme (phospholipase) are added (pH 4-6). This mixture goes to a reactor so that the enzymes can cleave the phospholipid bonds. The mixture is sent to a centrifuge to separate the sediment. This sediment represents around 3.5% total processed oil and is mainly composed of water, active enzymes, phosphorus, calcium and magnesium, which is industrially called lysogoma (FIG. 1).

Thus, the present inventor having full technical knowledge of the processes, found that the biggest drawback in the PLA (Phospholipase A) enzymatic treatment of degummed oil is the lysogoma (represented in FIG. 1), as this is stored in tanks until it reaches the volume for be discharged. The discharge of lysogoma is performed by specialized companies, which generates an additional cost to the process.

In research performed on the state of the art, several documents were identified describing methods and processes for the treatment and refining of vegetable oils, where we can highlight the following documents:

EP3601507 (Novozymes. 2017) describes the refining of vegetable oil wherein the phospholipids present in the vegetable oil are hydrolyzed and the oil is subsequently subjected to chemical refining.

BR102014021463 (Petrobras. 2014) describes a process for the treatment of acidic oils through an enzyme-catalyzed esterification reaction, which aims to make biodiesel production competitive, using low-cost and energy-saving raw materials, reducing the operational cost of production.

WO2015162307 (Rodriguez, Juan Garcia. 2014) describes a method for producing biodiesel from natural and recycled vegetable oils, applicable to oils with different degrees of acidity, which are alternately subjected to esterification in an acidic medium, if needed, followed in any case by transesterification in alkaline media.

EP3008200 (Renewable. 2013) describes methods and devices for economically producing a purified biodiesel product from raw materials. Some embodiments of the methods comprise the use of at least one crude raw material pretreatment process and a free fatty acid refining process prior to transesterification and the formation of biodiesel and crude glycerin.

EP1999241 (Cargill. 2006) describes a system and method of degumming a vegetable oil comprising mixing a feed stream under ultra-high shear conditions to provide a mixed stream, passing the mixed stream through a holding tank and separate the mixed stream into an aqueous stream and an oil stream.

These documents cited in the prior art describe various processes for the treatment and refining of vegetable oils intended for the production of biodiesel and other applications, however these documents do not describe or foresee a step for reusing the lysogoma generated in the PLA enzymatic treatment of the already degummed oil.

Therefore, the present inventor, being aware of the market processes described in the prior art, developed an additional step to the enzymatic process PLA (Phospholipase A) which allows for the reuse of lysogoma in the pre-treatment of already degummed oil in order to reduce the contents of phosphorus, calcium and magnesium and, consequently, improve the oil for the PLA enzymatic treatment step. By reusing lysogoma it is possible to improve the quality and increase the yield of the processed product, reducing production costs and catalyst consumption in the subsequent biodiesel transesterification reaction.

Therefore, the object of the present invention is a process for reusing lysogoma applied in the pre-treatment of vegetable oils for subsequent PLA enzymatic treatment and transesterification of biodiesel. In short, the use of lysogoma, which contains active enzymes, is presented as a pre-treatment as it aims to reduce the contents of phosphorus, calcium and magnesium present in the already degummed oil. This new process results in the reuse of a by-product with no commercial value helping to significantly reduce the contents of phosphorus, calcium and magnesium, which negatively interfere in the modification processes of vegetable oils, especially in the enzymatic treatment and transesterification steps for the production of biodiesel.

### SUMMARY OF THE INVENTION

A feature of the present invention is the process of reusing the lysogoma from the PLA (Phospholipase A) enzymatic treatment step in the already degummed oil. Since the lysogoma contains active enzymes and the capacity for molecular interactions, its reuse aims to improve the quality of the oil for the remaining stages.

A feature of the present invention is a lysogoma reuse process that provides for a reduction in the contents of phosphorus, calcium and magnesium in the degummed oil, which negatively interfere with the biodiesel transesterification process.

A feature of the present invention is a lysogoma reuse process that predict a technological application of a waste with no commercial value, helping to increase product yield and reduce production costs such as the amount of enzyme in enzymatic treatment and catalyst consumption in the subsequent biodiesel transesterification reaction.

### BRIEF DESCRIPTION OF THE FIGURES

FIG. 1 represents the lysogoma resulting from conventional PLA (Phospholipase A) enzymatic treatment.

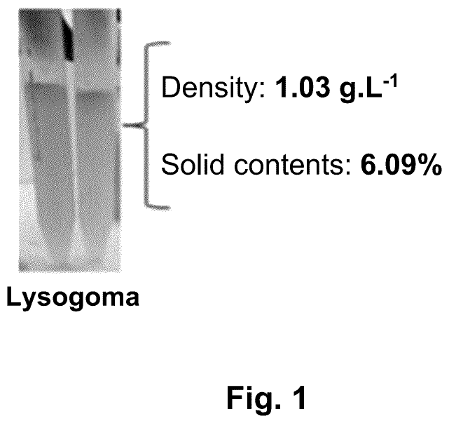

FIG. 2 shows steps of the conventional PLA process.

FIG. 3 shows the simplified scheme of the lysogoma reuse process for pre-treatment of already degummed oil, detailing the PLA enzymatic processing steps and the new pre-treatment process of lysogoma reuse.

### DETAILED DESCRIPTION OF THE INVENTION

The lysogoma reuse process applied in the pre-treatment of already degummed oils, object of the present invention, comprises a lysogoma reuse step, which still contains active enzymes originating from the PLA enzymatic treatment in the already degummed oil. Through a hydrolysis reaction (since there are active enzymes in the lysogoma) and through molecular interaction and drag force, the process of reusing the lysogoma aims to reduce the phosphorus, calcium and magnesium contents of the already degummed oil.

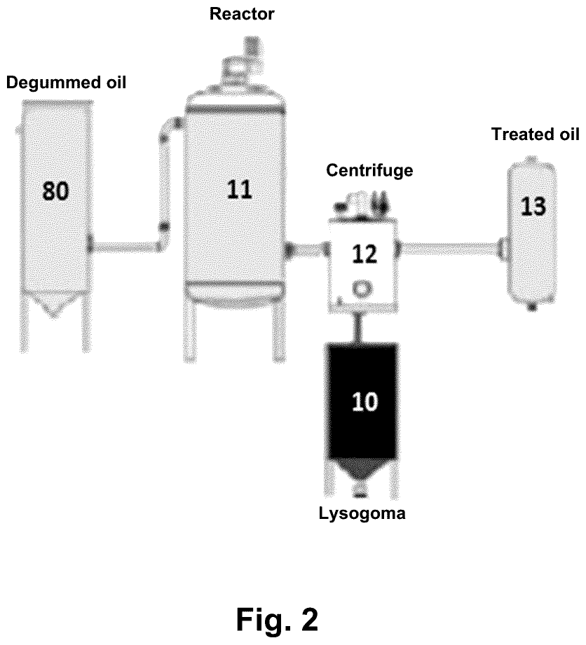

The conventional PLA process (hydrolysis by action of phospholipases A) occurs in the reaction step (11) obtained from the addition of the PLA enzyme and water (3.0 to 3.5%) proportional to the volume of already degummed oil entering the process (80). After a certain reaction time (11), the enzyme/oil mixture goes to the centrifugation step (12), which aims to remove undesirable constituents. The residue generated in this conventional process is called Lysogoma, which consists of water, phosphorus, calcium, magnesium and active phospholipase enzymes. FIG. 2 shows the steps of the conventional PLA process.

The lysogoma reuse process has the following steps:
The lysogoma (10) resulting from the PLA enzymatic treatment (11) (hydrolysis by action of phospholipases) of the degummed oil is returned in a continuous flow to a mixer (20) and reactor (30) for pre-treatment of the degummed oil (40), that is, the lysogoma (10) generated in the original PLA process is redirected to a new step (100) to be used in the pre-treatment of the degummed oil;
The degummed oil (40) is heated through a heat exchanger (50), between 50-70° C., for subsequent mixing (20) with the lysogoma (100). At this step, temperature conditions may vary according to the enzyme used in the PLA enzymatic process (11).

Next, the oil-lysogoma mixture goes to the reaction step, which takes place in a reactor (30). At this step, hydrolysis of phospholipid bonds occurs. Also due to molecular interaction and drag force, the contents of phosphorus, calcium and magnesium decrease.

Depending on the production line, the lysogoma (100) can be separated from the oil by a decantation (60) or centrifugation (70) operation to remove it from the processing line. After the degummed oil undergoes new pre-treatment, it is placed in a tank (80) wherein it undergoes conventional PLA enzymatic treatment (11) and centrifugation (12) to form the lysogoma (10) following the continuous flow of lysogoma formation. The lysogoma (100) is then removed from the oil, because it contains phosphorus, calcium and magnesium, as well as inactive enzymes that impair the other steps (13) of oil treatment and transesterification;

Thus, after the new lysogoma reuse process, the oil with low contents of phosphorus, calcium and magnesium goes to conventional PLA treatment and subsequently to the biodiesel transesterification step. It is worth mentioning that the steps may vary, depending on the processing plant, the direction or processing of these oils.

### Laboratory Test

After reusing the lysogoma, it was identified that the oil from this pre-treatment has a considerable reduction in the content of phosphorus, calcium and magnesium. It also has better performance in the following steps of PLA treatment and transesterification, as can be identified in Tables 1, 2, 3 and 4.

TABLE 1
Phosphorus content in the degummed oil (P001)

| SAMPLES | | | |
|---|---|---|---|
| Degummed oil | | | |
| Control | | PHOSPHORUS | |
| Holding Time | | (ppm) | |
| (hours) | Lysogoma | 71.7 | CONTENT |
| 0 | 3.5% | 39.3 (45.2%) | ↓ |
| 8 | 3.5% | 32.5 (54.7%) | |
| 16 | 3.5% | 30.4 (57.6%) | |
| 24 | 3.5% | 29.8 (58.4%) | |

TABLE 2
Calcium content in the degummed oil (P001)

| SAMPLES | | | |
|---|---|---|---|
| Degummed oil | | CALCIUM | |
| control | | (ppm) | |
| Holding Time | Lysogoma | 48.9 | CONTENT |
| 0 | 3.5% | 28.8 (41.1%) | ↓ |
| 8 | 3.5% | 33.6 (31.3%) | |
| 16 | 3.5% | 33.3 (31.9%) | |
| 24 | 3.5% | 32.8 (32.9%) | |

TABLE 3
Magnesium content in the degummed oil (P001)

| SAMPLES | | | |
|---|---|---|---|
| Degummed oil | | MAGNESIUM | |
| control | | (ppm) | |
| Holding Time | Lysogoma | 19.7 | CONTENT |
| 0 | 3.5% | 11.8 (40.1%) | ↓ |
| 8 | 3.5% | 10.3 (47.7%) | |
| 16 | 3.5% | 9.9 (49.7%) | |
| 24 | 3.5% | 9.9 (49.7%) | |

TABLE 4
Phosphorus, Calcium and Magnesium Content

| | P | Ca | Mg |
|---|---|---|---|
| Degummed oil | 66.65 | 23.85 | 8.71 |
| (Control) | | | |
| After 4 hours | 9.21 | 2.43 | <0.01 |
| of reaction / | 12.20 | 2.16 | <0.01 |
| 12 hours of | 12.14 | 2.09 | <0.01 |
| holding | | | |
| Reduction | 83.22% | 90.66% | 100% |

Thereby, a significant reduction in the contents of phosphorus, calcium and magnesium in the degummed oil was verified. However, these indexes may undergo some changes depending on the quality of the degummed oil and the condition of the lysogoma. In general, in some cases the results reach a reduction of more than 83% for phosphorus content, 90% for calcium content and up to 100% for magnesium content.

So, the percentage of reduction depends on several parameters, however it is worth highlighting that a significant reduction of these elements will always occur when using the new Lysogoma reuse process, which uses the residue from the PLA enzymatic process called Lysogoma in the pre-treatment of an oil already degummed.

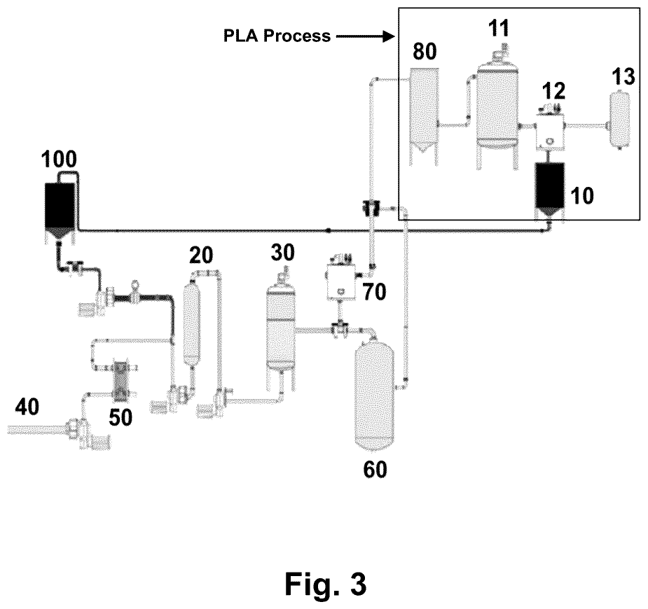

1. REUSE PROCESS OF THE LYSOGOMA APPLIED IN THE PRE-TREATMENT OF DEGUMMED VEGETABLE OILS FOR SUBSEQUENT ENZYME TREATMENT AND TRANSESTERIFICATION OF BIODIESEL, characterized in that it describes the following steps (FIG. 3):
a) the lysogoma (10), resulting from the PLA enzymatic treatment (11) (hydrolysis by action of phospholipases) of the already degummed oil is returned in a continuous flow to a mixer (20) and reactor (30) for pre-treatment of the degummed oil (100), that is, the lysogoma (10) generated in the original process is redirected to a new step (100) of pre-treatment of the degummed oil;
b) the degummed oil (40) is heated by a heat exchanger (50) between 50-70° C., for subsequent mixing (20) with the lysogoma (100), and in this stage the temperature conditions may vary according to with the enzyme used;
c) then the oil-lysogoma mixture goes to the reaction step, which takes place in a reactor (30). Where enzymatic hydrolysis of phospholipid bonds occurs, and by molecular interaction and drag force, there is a decrease in phosphorus, calcium and magnesium content;
d) as the production line, the lysogoma (100) can be separated from the oil by a decantation (60) or centrifugation (70) operation to remove it from the processing line, because it contains constituents such as: phosphorus, calcium and magnesium, in addition to inactive enzymes that impair the other processing steps (treatment and transesterification), so that after its removal, the degummed oil is placed in a tank (80) where it undergoes PLA enzymatic treatment (11) and centrifugation (12) to form the lysogoma (10) following the continuous flow of lysogoma formation (100);
e) thus, after the reuse process (100), the oil with low levels of phosphorus, calcium and magnesium goes to conventional PLA enzymatic treatment and subsequent biodiesel transesterification step.
